(19) United States
(12) Patent Application Publication
(10) Pub. No.: US 2021/0346615 A1
(43) Pub. Date: Nov. 11, 2021

(54) DEVICES AND METHODS FOR DELIVERING A SUBSTANCE TO A BODY CAVITY UTILIZING BLOW-FILL-SEAL TECHNOLOGY

(71) Applicant: SipNose Ltd., Yokneam (IL)

(72) Inventors: Daniel SHAHAF, M.P. Emek Ha-Yarden (IL); Iris SHICHOR, Zichron Ya'akov (IL); Liron Shavit HADAR, Haifa (IL)

(73) Assignee: SipNose Ltd., Yokneam (IL)

(21) Appl. No.: 17/354,771

(22) Filed: Jun. 22, 2021

Related U.S. Application Data
(63) Continuation-in-part of application No. PCT/IB2021/051834, filed on Mar. 4, 2021.
Application No. PCT/IB2021/051834 is a continuation-in-part of application No. 16/809,994, filed on Mar. 5, 2020.
Application No. 16/809,994 is a continuation-in-part of application No. 16/810,096, filed on Mar. 5, 2020.
Application No. 16/810,096 is a continuation-in-part of application No. 15/982,996, filed on May 17, 2018.
Application No. 16/810,096 is a continuation-in-part of application No. 15/982,630, filed on May 17, 2018.
Application No. 15/982,630 is a continuation-in-part of application No. 14/733,143, filed on Jun. 8, 2015, now Pat. No. 11,116,914.
Application No. 15/982,996 is a continuation-in-part of application No. 14/733,143, filed on Jun. 8, 2015, now Pat. No. 11,116,914.
Application No. 16/809,994 is a continuation-in-part of application No. 14/433,048, filed on Apr. 2, 2015.
(60) Provisional application No. 63/043,805, filed on Jun. 25, 2020.
(60) Provisional application No. 62/526,386, filed on Jun. 29, 2017.
(60) Provisional application No. 62/507,816, filed on May 18, 2017.
(60) Provisional application No. 62/117,986, filed on Feb. 19, 2015.
(60) Provisional application No. 62/077,246, filed on Nov. 9, 2014.
(60) Provisional application No. 61/868,614, filed on Aug. 22, 2013.
(60) Provisional application No. 61/868,627, filed on Aug. 22, 2013.

(30) Foreign Application Priority Data
Dec. 16, 2013 (DE) 2020131057150

(51) Int. Cl. A61M 11/00; A61M 15/00

(52) U.S. Cl. CPC A61M 11/007; A61M 2202/064; A61M 15/0013; A61M 15/0035; A61M 2205/02; A61M 2210/0618; A61M 2210/0612; A61M 2210/0662; A61M 2210/1475; A61M 2210/065; A61M 2210/1089; A61M 2202/0208; A61M 2202/025; A61M 2202/0225; A61M 2202/0291; A61M 2202/0275; A61M 2202/04; A61M 11/001

(57) ABSTRACT

A device is provided for delivering one or more substances within at least one body cavity. The device includes at least one pierceable vial comprising V_{sub} [ml or mg] of the substances; the vial having at least one fluid discharging outlet port, configured for placement in proximity to the body cavity. The pierceable vial is configured to interface in a sealable manner with at least one pressurized fluid at volume V_{PF} [ml] and pressure P_{PF} [barg]. The device, upon actuation, permits the pressurized fluid to enter into the pierceable vial via the at least one fluid inlet port, entrains the substances, and delivers the substances through the fluid discharging outlet port to within the body cavity in the form of at least one selected from a group consisting of drops, droplets, stream, aerosol and any combination thereof.

### CROSS REFERENCE TO RELATED APPLICATIONS

This application is a Continuation-in-Part of U.S. application Ser. No. 16/809,994, filed on Mar. 5, 2020, which is a Continuation-in-Part of U.S. application Ser. No. 15/982,996 filed on May 17, 2018, which is a Continuation-in-Part of U.S. application Ser. No. 14/733,143 filed on Jun. 8, 2015, which claims the benefit of and priority to U.S. Provisional Application No. 62/117,986 filed on Feb. 19, 2015, and U.S. Provisional Application No. 62/077,246 filed on Nov. 9, 2014. U.S. application Ser. No. 15/982,996 claims further priority to U.S. Provisional Application No. 62/526,386 filed on Jun. 29, 2017.

U.S. application Ser. No. 16/809,994 is also a Continuation-in-Part of U.S. application Ser. No. 16/810,096 filed Mar. 5, 2020, which is a Continuation-in-Part of U.S. application Ser. No. 15/982,630 filed on May 17, 2018, which is a Continuation-in-Part of U.S. application Ser. No. 14/733,143 filed on Jun. 8, 2015, which claims the benefit of and priority to U.S. Provisional Application No. 62/117,986 filed on Feb. 19, 2015, and U.S. Provisional Application No. 62/077,246 filed on Nov. 9, 2014. U.S. application Ser. No. 15/982,630 further claims priority to U.S. Provisional Application No. 62/507,816 filed on May 18, 2017.

Further, U.S. application Ser. No. 16/809,994 is a Continuation-in-Part of U.S. application Ser. No. 14/433,048 filed on Apr. 2, 2015, which is a National Phase Entry of PCT/IL2014/050752 filed on Aug. 21, 2014, which claims the benefit of and priority to U.S. Provisional Application No. 61/868,614 filed on Aug. 22, 2013, and U.S. Provisional Application No. 61/868,627 filed on Aug. 22, 2013, and further priority to German Application No. 2020131057150 filed on Dec. 16, 2013.

Additionally, this application claims priority to and the benefit of U.S. Provisional Application No. 63/043,805 filed on Jun. 25, 2020.

This application is a Continuation-in-Part of International Application No. PCT/IB2021/051834 filed on Mar. 4, 2021.

The contents of the above applications are all incorporated by reference as if fully set forth herein in their entirety.

### FIELD OF THE INVENTION

The present invention generally pertains to a system and new methods for utilizing BFS and Form Fill Seal, FFS containers (and alike) in delivering substance to a natural orifice of the body.

### BACKGROUND OF THE INVENTION

Blow-Fill-Seal (BFS) or FFS technology is a manufacturing technique used to produce small (0.1 ml) and large (and up to 500 ml) liquid-filled containers. The basic concept of blow-fill-seal and form-fill-seal (referred to interchangeably hereinafter as BFS) is that a container is formed, filled and sealed in a continuous process without human intervention in a sterile or aseptic enclosed area. Thus, this technology can be used to sterile or aseptically packaging and manufacturing of pharmaceutical liquid dosage forms.

There are several ways of manufacturing. According to one method, the processes begins with as pharmaceutical grade plastic resin is vertically heat-extruded through a circular throat to form a hanging tube (parison). This extruded tube is then enclosed within a two-part mold and the tube is cut above the mold. The mold zone, or a sterile filling space, where filling needles (mandrels) are lowered and used to inflate the plastic to form a container within the mold. Following formation of the container, the mandrel is used to fill the container with the liquid. Following filling, the mandrels are retracted and a secondary top mold seals the container. All actions take place within the sterile/aseptic enclosed area, a sterile shrouded chamber within the machine. The product can then be discharged to a non-sterile area for labeling, packaging and distribution.

BFS technology reduces personnel intervention, making it a more robust method for aseptic or sterile packaging of pharmaceuticals. BFS is used for the filling of vials for parenteral preparations and infusion, eye drops, and inhalation products. Generally, the containers are made of polyethylene and polypropylene or potentially other plastic or nylon materials.

Common methods of extracting the substance enclosed within the BFS includes squeezing and pressing on the BFS such that the substance stored therewithin is mechanically “pressed-out” of the BFS (usually used in eyedrops). Such squeezing is neither accurate nor reproducible to provide an exact dose each squeeze. Also, in cases were improved absorption into the tissue surface is preferable or needed, release of relatively small droplets of liquid is highly advantages in oppose to release of 2-3 very large drops from the BFS, as it results in large surface area thus improved absorption.

In most treatment, an accurate and reproducible dosing is required. Thus, it is therefore a long felt need to provide a system which provides an efficient delivery of a substance out of the BFS.

### SUMMARY OF THE INVENTION

It is one object of the present invention to provide a device for delivering either one or more substances within at least one body cavity, characterized by at least one pierceable vial comprising V_{sub} [ml or mg] of said substances; said vial having at least one fluid discharging outlet port, configured for placement in proximity to said body cavity; said pierceable vial configured to interface in a sealable manner with at least one pressurized fluid at volume V_{PF} [ml] and pressure P_{PF} [barg];

wherein said device, upon actuation, enables said pressurized fluid to enter into said pierceable vial via at least one said fluid inlet port, entrains said substances, and delivered through via said fluid discharging outlet port to within said body cavity in the form of at least one selected from a group consisting of drops, droplets, stream, aerosol and any combination thereof.

It is another object of the present invention to provide the device as defined above, wherein said pressurized fluid is enclosed within at least one pressurized fluid chamber.

It is another object of the present invention to provide the device as defined above, wherein said vial is selected from a group consisting of pierceable container, a blow-fill-seal and a form-fill-seal, any breakable containers, any puncturable container and any combination thereof.

It is another object of the present invention to provide the device as defined above, additionally comprising an activation mechanism being at least one valve adapted, upon actuation of the same, to enable the entry of said pressurized fluid into said pierceable vial.

It is another object of the present invention to provide the device as defined above, additionally comprising an activation mechanism being at least one valve adapted, upon actuation of the same, to enable the release of said pressurized fluid from said pressurized fluid chamber.

It is another object of the present invention to provide the device as defined above, additionally comprising an activation mechanism being at least one valve adapted, upon actuation of the same, to enable the release of said pressurized fluid from said pressurized fluid chamber and entry of said pressurized fluid into said pierceable vial.

It is another object of the present invention to provide the device as defined above, additionally comprising an activation mechanism being at least one breakable membrane, such that when the pressure of said pressurized fluid, P_{PF}, reaches a predetermined value, said breakable membrane breaks and enable the entry of said pressurized fluid into said pierceable vial.

It is another object of the present invention to provide the device as defined above, additionally comprising an activation mechanism being at least one breakable membrane, such that when the pressure of said pressurized fluid, P_{PF}, reaches a predetermined value, said breakable membrane breaks and enable the release of said pressurized fluid from said pressurized fluid chamber.

It is another object of the present invention to provide the device as defined above, additionally comprising an activation mechanism being at least one breakable membrane, such that when the pressure of said pressurized fluid, P_{PF}, reaches a predetermined value, said breakable membrane breaks and enable the release of said pressurized fluid from said pressurized fluid chamber and the entry of said pressurized fluid into said pierceable vial.

It is another object of the present invention to provide the device as defined above, additionally comprising an activation mechanism being at least one piercing element adapted to pierce said pierceable vial.

It is another object of the present invention to provide the device as defined above, wherein said at least one chamber is a container adapted to hold said pressured fluid at said P_{PF} for prolong periods of time.

It is another object of the present invention to provide the device as defined above, wherein said vial is made of material selected from a group consisting of high density polyethylene and\or polypropylene and any other plastic resin.

It is another object of the present invention to provide the device as defined above, wherein said pressurized fluid is breath exhaled from the user of said device.

It is another object of the present invention to provide the device as defined above, wherein said pressurized fluid is provided by external fluid pump.

It is another object of the present invention to provide the device as defined above, wherein said V_{sub} is in a range of about 0.001 to about 7 ml or 0.001 mg to 7 g.

It is another object of the present invention to provide the device as defined above, wherein said V_{PF} is in a range of 0.1 to 50 ml.

It is another object of the present invention to provide the device as defined above, wherein said P_{PF} is in a range of about 0 to about 10 barg.

It is another object of the present invention to provide the device as defined above, wherein said at least one fluid inlet port is in a range of 0.01 to 30 mm.

It is another object of the present invention to provide the device as defined above, wherein said at least one fluid discharging outlet port is in a range of 0.01 to 30 mm.

It is another object of the present invention to provide the device as defined above, wherein said chamber is a syringe-like device.

It is another object of the present invention to provide the device as defined above, wherein said pressurized fluid is compressed within said chamber by means of at least one piston.

It is another object of the present invention to provide the device as defined above, additionally comprising at least one piercing element coupled to said piston; said piercing element is adapted to pierce said pierceable vial.

It is another object of the present invention to provide the device as defined above, wherein said release of said substances along with said pressurized fluid is performed within a period of time smaller than 3 seconds (dT).

It is another object of the present invention to provide the device as defined above, wherein said release of said substances along with said pressurized fluid is performed within a short period of time, <500 milliseconds (dT).

It is another object of the present invention to provide the device as defined above, wherein at least one of the following is true:
a. said body cavity is selected from a group consisting of nasal cavity, the mouth, the throat, an ear, the eye, the vagina, the rectum, the urethra, and any combination thereof.
b. said pressurized fluid is selected from a group consisting of air, nitrogen, oxygen, carbon dioxide, helium, neon, xenon, nitric oxide and any combination thereof;
c. during dispensing of said at least one substance, a mixture of said predetermined volume V_{gas} [ml] of said pressurized fluid with said predetermined volume V_{sub} [ml or mg] of said substance entrained within it forms a plume of aerosol; said aerosol having a predetermined distribution, said distribution being either homogeneous or heterogeneous, said heterogeneous distribution is selected from a group consisting of: an arbitrary distribution, a distribution in which the density of said at least one substance within said mixture follows a predetermined pattern, and any combination thereof; characteristics of said aerosol selected from a group consisting of: particle size, particle shape, particle distribution, and any combination thereof, are determinable from characteristics of said device selected from a group consisting of: said predetermined volume of said pressurized fluid, said predetermined volume of said substance, said predetermined pressure of said pressurized fluid, said predetermined orifice size, and any combination thereof;
d. at least one said substance is selected from a group consisting of a fluid, a liquid, a powder, an aerosol, a slurry, a gel, a suspension and any combination thereof; and
e. at least one said substance is stored under either an inert atmosphere or under vacuum to prevent reactions during storage.

It is another object of the present invention to provide the device as defined above, wherein said chamber is made of material being high barrier film.

It is another object of the present invention to provide the device as defined above, wherein said high barrier film is high barrier Aluminum film.

It is another object of the present invention to provide a method for delivering either one or more substances within at least one body cavity, wherein said method comprising steps of:
a. providing a device being characterized by at least one pierceable vial comprising V_{sub} [ml or mg] of said substances; said vial having at least one fluid discharging outlet port; said pierceable vial configured to interface in a sealable manner with at least one pressurized fluid at volume V_{PF} [ml] and pressure P_{PF} [barg];
b. placing said device in proximity to said body cavity; and
c. activating said device; thereby enabling said pressurized fluid to enter into said pierceable vial via at least one said fluid inlet port, and to entrain said at least one substance, and delivered through via said fluid discharging outlet port into said body cavity in the form of at least one selected from a group consisting of drops, droplets, stream, aerosol and any combination thereof.

It is another object of the present invention to provide the method as defined above, wherein said pressurized fluid is enclosed within at least one pressurized fluid chamber.

It is another object of the present invention to provide the method as defined above, wherein said vial is selected from a group consisting of pierceable container, a blow-fill-seal and a form-fill-seal, any breakable containers, any puncturable container and any combination thereof.

It is another object of the present invention to provide the method as defined above, additionally comprising an activation mechanism being at least one valve adapted, upon actuation of the same, to enable the entry of said pressurized fluid into said pierceable vial.

It is another object of the present invention to provide the method as defined above, additionally comprising an activation mechanism being at least one valve adapted, upon actuation of the same, to enable the release of said pressurized fluid from said pressurized fluid chamber.

It is another object of the present invention to provide the method as defined above, additionally comprising an activation mechanism being at least one valve adapted, upon actuation of the same, to enable the release of said pressurized fluid from said pressurized fluid chamber and entry of said pressurized fluid into said pierceable vial.

It is another object of the present invention to provide the method as defined above, additionally comprising an activation mechanism being at least one breakable membrane, such that when the pressure of said pressurized fluid, P_{PF}, reaches a predetermined value, said breakable membrane breaks and enable the entry of said pressurized fluid into said pierceable vial.

It is another object of the present invention to provide the method as defined above, additionally comprising an activation mechanism being at least one breakable membrane, such that when the pressure of said pressurized fluid, P_{PF}, reaches a predetermined value, said breakable membrane breaks and enable the release of said pressurized fluid from said pressurized fluid chamber.

It is another object of the present invention to provide the method as defined above, additionally comprising an activation mechanism being at least one breakable membrane, such that when the pressure of said pressurized fluid, P_{PF}, reaches a predetermined value, said breakable membrane breaks and enable the release of said pressurized fluid from said pressurized fluid chamber and the entry of said pressurized fluid into said pierceable vial.

It is another object of the present invention to provide the method as defined above, additionally comprising an activation mechanism being at least one piercing element adapted to pierce said pierceable vial.

It is another object of the present invention to provide the method as defined above, wherein said at least one chamber is a container adapted to hold said pressured fluid at said P_{PF} for prolong periods of time.

It is another object of the present invention to provide the method as defined above, wherein said vial is made of material selected from a group consisting of high density polyethylene and\or polypropylene and any other plastic resin.

It is another object of the present invention to provide the method as defined above, wherein said pressurized fluid is breath exhaled from the user of said device.

It is another object of the present invention to provide the method as defined above, wherein said pressurized fluid is provided by external fluid pump.

It is another object of the present invention to provide the method as defined above, wherein said V_{sub} is in a range of about 0.001 to about 7 ml or 0.001 mg to 7 g.

It is another object of the present invention to provide the method as defined above, wherein said V_{PF} is in a range of 0.1 to 50 ml.

It is another object of the present invention to provide the method as defined above, wherein said P_{PF} is in a range of about 0 to about 10 barg.

It is another object of the present invention to provide the method as defined above, wherein said at least one fluid inlet port is in a range of 0.01 to 30 mm.

It is another object of the present invention to provide the method as defined above, wherein said at least one fluid discharging outlet port is in a range of 0.01 to 30 mm.

It is another object of the present invention to provide the method as defined above, wherein said chamber is a syringe-like device.

It is another object of the present invention to provide the method as defined above, wherein said pressurized fluid is compressed within said chamber by means of at least one piston.

It is another object of the present invention to provide the method as defined above, additionally comprising at least one piercing element coupled to said piston; said piercing element is adapted to pierce said pierceable vial.

It is another object of the present invention to provide the method as defined above, wherein said release of said substances along with said pressurized fluid is performed within a short period of time smaller than 3 seconds (dT).

It is another object of the present invention to provide the method as defined above, wherein said chamber is made of material being high barrier film.

It is another object of the present invention to provide the method as defined above, wherein said high barrier film is high barrier Aluminum film.

It is lastly an object of the present invention to provide the method as defined above, wherein at least one of the following is true:
a. said body cavity is selected from a group consisting of nasal cavity, the mouth, the throat, an ear, the eye, the vagina, the rectum, the urethra, and any combination thereof.
b. said pressurized fluid is selected from a group consisting of air, nitrogen, oxygen, carbon dioxide, helium, neon, xenon, nitric oxide and any combination thereof;
c. during dispensing of said at least one substance, a mixture of said predetermined volume V_{gas} [ml] of said pressurized fluid with said predetermined volume V_{sub} [ml or mg] of said substance entrained within it forms a plume of aerosol; said aerosol having a predetermined distribution, said distribution being either homogeneous or heterogeneous, said heterogeneous distribution is selected from a group consisting of: an arbitrary distribution, a distribution in which the density of said at least one substance within said mixture follows a predetermined pattern, and any combination thereof; characteristics of said aerosol selected from a group consisting of: particle size, particle shape, particle distribution, and any combination thereof, are determinable from characteristics of said device selected from a group consisting of: said predetermined volume of said pressurized fluid, said predetermined volume of said substance, said predetermined pressure of said pressurized fluid, said predetermined orifice size, and any combination thereof;
d. at least one said substance is selected from a group consisting of a fluid, a liquid, a powder, an aerosol, a slurry, a gel, a suspension and any combination thereof; and
e. at least one said substance is stored under either an inert atmosphere or under vacuum to prevent reactions during storage.

### BRIEF DESCRIPTION OF THE DRAWINGS

The invention is herein described, by way of example only, with reference to the accompanying drawings, wherein:

FIGS. 1-3 show a first embodiment of the present invention;

FIGS. 4-6 show a second embodiment of the present invention;

FIGS. 7-9a show a third embodiment of the present invention;

FIG. 9b illustrates another embodiment of the air tight container enclosing the pressurized fluid;

FIGS. 10A-B shows an embodiment of the present invention, with FIG. 10A showing an exploded view of the device and FIG. 10B showing the device fully assembled;

FIGS. 11A-C shows another embodiment of the present invention, with FIG. 11A showing the device, FIG. 11B showing a cross section of the device and FIG. 11C showing an enlarged view of a portion of the device;

FIGS. 12A-D shows another embodiment of the present invention, with FIG. 12A showing a cross-section of the device, FIG. 12B showing an enlarged view a portion of the device, FIG. 12C showing the exterior of the nosepiece and FIG. 12D showing the device from the top;

FIGS. 13A-C shows another embodiment of the present invention, after activation, with the device shown in FIG. 13A, a cross section of the device in FIG. 13B and an enlarged view a portion of the device in FIG. 13C;

FIGS. 14A-G show another embodiment of the device, with FIG. 14A showing a side view of a pre-used device carrying a BFS or FFS, FIG. 14B showing a cross section of the same, FIGS. 14C and 14D depicting the device when connected to a BFS, FIG. 14E showing the same when the device is ready to use, FIG. 14F showing connection of the BFS or FFS to the device; and FIG. 14G showing the device after activation;

FIGS. 15A-E show another embodiment of the present invention, with FIG. 15A illustrating a front view of a pre-used device carrying a BFS or FFS and cross sections of the same; FIG. 15B showing securing of the BFS or FFS to the device and breaking the cap; FIG. 15C presenting a cross-section view; FIG. 15D showing a button at the base of the device being pushed; FIG. 15E showing pressurized fluid flowing from the container to the BFS or FFS and carrying the drug outward;

FIGS. 16A-16B illustrating a device being another embodiment of the present invention, a front and exploded view, respectively;

FIGS. 17A-17E shows same device in various modes of operation; and

FIG. 18 depict two views of the device interconnected to a safety latch according to an embodiment of the present invention.

### DETAILED DESCRIPTION OF THE PREFERRED EMBODIMENTS

The following description is provided, alongside all chapters of the present invention, so as to enable any person skilled in the art to make use of the invention and sets forth the best modes contemplated by the inventor of carrying out this invention. Various modifications, however, will remain apparent to those skilled in the art, since the generic principles of the present invention have been defined specifically to provide a device for delivering substances utilizing a BFS or FFS technology.

In the present invention, a combination of parameters and forces such as pressure, gas/air volume orifice diameter enable the enhanced delivery of a substance enclosed within a BFS or FFS.

The term ‘pierce’ refers hereinafter to any action of piercing or breaking or cutting or any other action enabling the penetration into the pierceable element. According to the present invention, the vial is a pierceable vial that enables penetration of fluid (e.g., pressurized fluid) therewithin. The piercing could be provided by means of a sharp element penetrating the same, a breakable membrane/sheath/septum, opening of a valve to enable passage therewithin.

The term ‘high barrier films’ refers hereinafter to any materials in flexible packaging laminations that prevent the permeation of water, water vapor, oil, oxygen, aroma, flavor, gas, or light. Such provision is enabled by low permeability of the film. In some embodiments the film is made of high-density Aluminum films. Thus, according to one embodiment of the present invention, the pressurized gas enclosed within high barrier film that can maintain the pressure of the compressed gas for prolong period of times without leakage therefrom.

The term ‘substance’ hereinafter refers to any substance capable of flowing. Such a substance can be a granular material, including a powder; a liquid; a gel; a slurry; a suspension; and any combination thereof. The term further refers to one or more members of a group consisting of proteins; stem-cells; cells, organs, portions, extracts, and isolations thereof; macro-molecules; RNA or other genes and proteins-encoding materials; neurotransmitters; receptor antagonists; biologic response modifiers; hormones; Ketamine; commercially available by Lilly (US) Baqsimi product; biologic response modifiers; Glucagon; substrates to treat one of eth followings: anaphylaxis, Parkinson, seizures and opioid overdose; epinephrine; atropine; metoclopramide; commercially available Naloxone or Narcan products; Esketamine (Spravato); Radicava [edaravone]; Ingrezza [valbenazine]; Austedo [deutetrabenazine]; Ocrevus [ocrelizumab]; Xadago [safinamide]; Spinraza [nusinersen]; Zinbryta [daclizumab]; Nuplazid [pimavanserin]; Aristada [aripiprazole lauroxil]; Vraylar [cariprazine]; Rexulti [brexpiprazole]; Aptiom [eslicarbazepine acetate]; Vizamyl [flutemetamol F18 injection]; Brintellix [vortioxetine]; Tecfidera [dimethyl fumarate]; Dotarem [gadoterate meglumine]; Antibody mediated brain targeting drug delivery including aducanumab, gantenerumab, bapineuzumab, solanezumab, ofatumumab CD20, BIIB033, LCN2, HMGB1; insulin; oxytocin; orexin-A; leptin; benzodiazepine i.e. midazolam; naloxone; perillyl alcohol; camptothecin; phytochemicals including curcumin and chrysin; nucleotides; olanzapine; risperidone; Venlafaxin; GDF-5; zonisamide; ropinirole; plant-originated and synthetically-produced terpenes and cannabinoids, including THC and CBD; valproric acid; rivastigmine; estradiol; topiramate or an equivalent preparation comprising CAS No. 97240-79-4; MFSD2 or MFSD2A or sodium-dependent lysophosphatidylcholine symporter; and any esters, salts, derivatives, mixtures, combinations thereof, with or without a carrier, liposomes, lyophilic or water-miscible solvents, surfactants, cells, cells fractions, at a therapeutically effective concentration.

The term ‘gas’ refers to any fluid that can be readily compressed. Gases as used herein include, but are not limited to, air, nitrogen, oxygen, carbon dioxide, helium, neon, xenon, nitric oxide and any combination thereof. Devices charged by hand will typically use air as the carrier gas.

The term ‘fluid’ refers to any substance or mixtures of substances that continually deforms (flows) under an applied shear stress, or external force. This term refers to gas, liquids, particulate or granulated solids (powders), aerosols, and any mixtures and combinations thereof.

The term ‘about’ refers hereinafter to a range of 25% below or above the referred value.

The terms “body orifice” and “body cavity” are interchangeably refer to one or more of the followings: nasal cavity, a mouth, a throat, an ear, a vagina, a rectum, a urethra, and any combination thereof.

All pressures herein are gauge pressures, relative to atmospheric pressure. Pressure units will be written herein using the standard abbreviation for “gauge’, namely, “g”. For example, atmospheric pressure is 0 barg and a pressure of 1 bar above atmospheric is 1 barg.

It is thus, one object of the present invention to provide a device for delivering either one or more substances within at least one body cavity, characterized by at least one pierceable vial comprising V_{sub} [ml or mg] of said substances; said vial having at least one fluid discharging outlet port, configured for placement in proximity to said body cavity; said pierceable vial configured to interface in a sealable manner, via at least one activation mechanism (activator) with at least one pressurized fluid chamber; said pressurized fluid chamber configured to contain pressurized fluid at volume V_{PF} [ml] and pressure P_{PF} [barg]; wherein said activation mechanism, upon actuation, enables said pressurized fluid to flow from said chamber, via said fluid inlet port, entrains said substances, erupts via said fluid discharging outlet port to within said body cavity in the form of at least one selected from a group consisting of drops, droplets, stream, aerosol and any combination thereof.

It is another object of the present invention to provide the device as defined above, wherein said vial is selected from a group consisting of pierceable container, a blow-fill-seal and a form-fill-seal, any breakable containers, any puncturable container and any combination thereof.

It is another object of the present invention to provide the device as defined above, wherein said at least one chamber is a container adapted to hold said pressured fluid at said P_{PF} for prolong periods of time.

It is another object of the present invention to provide the device as defined above, wherein at least one of the following is true:
a. said body cavity is selected from a group consisting of nasal cavity, the mouth, the throat, an ear, the eye, the vagina, the rectum, the urethra, and any combination thereof.
b. said pressurized gas is selected from a group consisting of air, nitrogen, oxygen, carbon dioxide, helium, neon, xenon, nitric oxide and any combination thereof;
c. during dispensing of said at least one substance, a mixture of said predetermined volume V_{gas} [ml] of said pressurized gas with said predetermined volume V_{sub} [ml or mg] of said substance entrained within it forms a plume of aerosol; said aerosol having a predetermined distribution, said distribution being either homogeneous or heterogeneous, said heterogeneous distribution is selected from a group consisting of: an arbitrary distribution, a distribution in which the density of said at least one substance within said mixture follows a predetermined pattern, and any combination thereof; characteristics of said aerosol selected from a group consisting of: particle size, particle shape, particle distribution, and any combination thereof, are determinable from characteristics of said device selected from a group consisting of: said predetermined volume of said pressurized gas, said predetermined volume of said substance, said predetermined pressure of said pressurized gas, said predetermined orifice size, and any combination thereof;
d. at least one said substance is selected from a group consisting of a gas, a liquid, a powder, an aerosol, a slurry, a gel, a suspension and any combination thereof; and
e. at least one said substance is stored under either an inert atmosphere or under vacuum to prevent reactions during storage.

It is another object of the present invention to provide the device as defined above, wherein said vial is made of material selected from a group consisting of high-density polyethylene and\or polypropylene and any other plastic resin.

It is another object of the present invention to provide the device as defined above, wherein the chamber enclosing the pressurized fluid is made of material being high barrier film.

It is another object of the present invention to provide the device as defined above, wherein said high barrier film is high barrier Aluminum film.

The present invention teaches a device for delivering a predetermined amount of a substance, preferably comprising a therapeutic compound or combination of therapeutic compounds, such as but not limited to medications into a body orifice of a subject, the orifice comprising any of the body's natural orifices, including a nostril, the mouth, the ear, the throat, the urethra, the vagina, the rectum and any combination thereof.

The drugs to be applied could be, but are not limited to, pharmaceuticals, natural compounds, biologics, hormones, peptides, proteins, viruses, cells, stem cells and any combination thereof.

However, it should be emphasized that the device can be provided alone as well as in combination with a capsule.

In some cases, the capsule would be provided with a known medicament within the same and in other cases the capsule would be ‘filled’ with the medicament just before use.

In some embodiments of the present invention, the device operating characteristics and the substance characteristics can be jointly optimized to maximize uptake of the substance at the desired site. In preferred variants of such embodiments, uptake is further optimized by exploiting synergies between delivery characteristics generated by the device and by the formulation or composition of the delivered material

In some embodiments, the substance comprises one or more agents to optimize delivery through the mucosal membrane by means of mucoadhesive agent and/or a permeability enhancer agent and/or a particulate formulation in the nano-particle or macro-particle range, and any combination thereof. In such embodiments, the combination of the device and substance enhance the delivery of the active agent to the target area (nasal epithelium and more specifically olfactory epithelium) and from there to the target tissue (for example the brain).

Furthermore, according to some embodiments of the present invention, a new method of utilizing blow-fill-seal (BFS) or Form Fill Seal (FFS) technology to deliver substances is offered. The commonly used practice is squeeze said BFS or FFS to enable dripping of the substance therefrom. The present invention discloses a new method utilizing pressurized fluid (namely, pressurized gas) to entrain the substance within said BFS or FFS and deliver the same. Utilizing the pressure as a driving force and the air as a carrier allows the material to be released from BFS or FFS and into the desired target tissue.

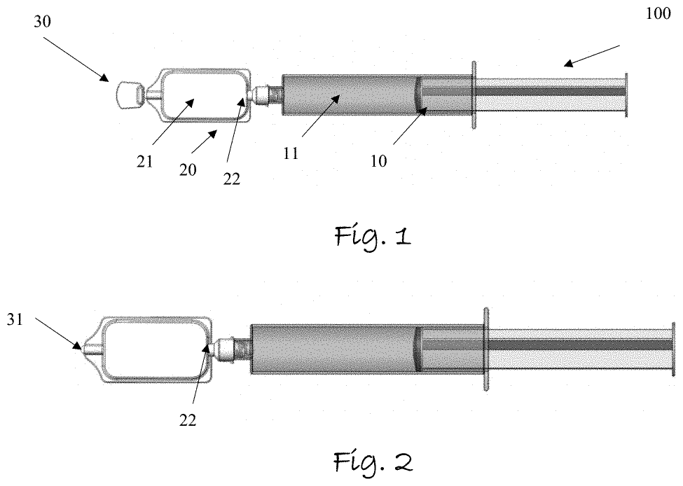

Reference is now made to FIGS. 1-3 illustrating a device according to one embodiment of the present invention in which a pierceable vial (e.g., BFS or FFS).

It should be noted that pierceable vial could be a blow-fill-seal and a form-fill-seal vial or any breakable containers (that breaks under mechanical pressure; e.g., the pressurized gas) or any puncturable container (that breaks by a sharp element; e.g., needle).

In this embodiment, a syringe 100 with a piston 10 is coupled to a BFS or FFS 20. The BFS or FFS 20 comprises a cover 30 breakably coupled thereto.

The syringe encloses at least one fluid (e.g., gas) 11 that will be pressurized during activation.

The fluid could be air, nitrogen, oxygen, carbon dioxide, helium, neon, xenon, nitric oxide and any combination thereof. The volume of the fluid could be in the ranges of 0.1 to 50 ml.

The BFS or FFS 20 encloses at least one substance (medicament) that after activation would be released. The volume of the substance is in a range of about 0.001 to about 7 ml or 0.001 mg to 7 g.

The BFS or FFS comprises at least one breakable membrane 22 such that upon activation and release of the pressurized fluid (as will be disclosed herein after), said membrane will break and enable the entrance of the pressurized fluid into the BFS or FFS, entrain the substance and deliver the same.

FIG. 1 illustrates the syringe 100 before activation thereof.

In FIG. 2 the cover 30 has been remove, revealing at least one orifice 31 of the BFS or FFS 20. Next, the piston compresses the fluid 21 enclosed therewithin to pressures in the ranges of about 0 to about 10 barg.

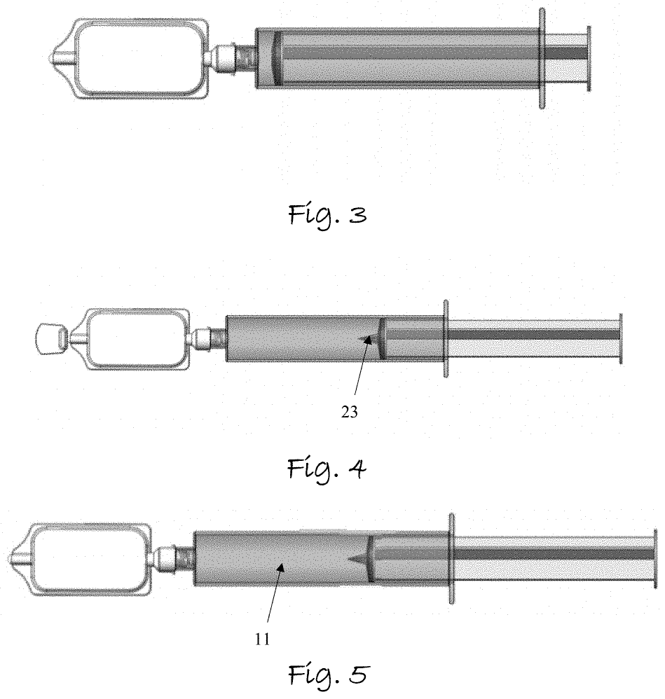

In FIG. 3 the piston is moved in an air-tight manner within the syringe and pressurizes the fluid to a predefine pressure. Once the pressure of the fluid reaches said predefined pressure, said membrane 22 breaks and the pressurized fluid enters the BFS or FFS and entrains the substance to be delivered.

Reference is now made to FIGS. 4-6 illustrating another embodiment of the present invention. In this embodiment, the piston has a piercing end 23 adapted to pierce the BFS or FFS.

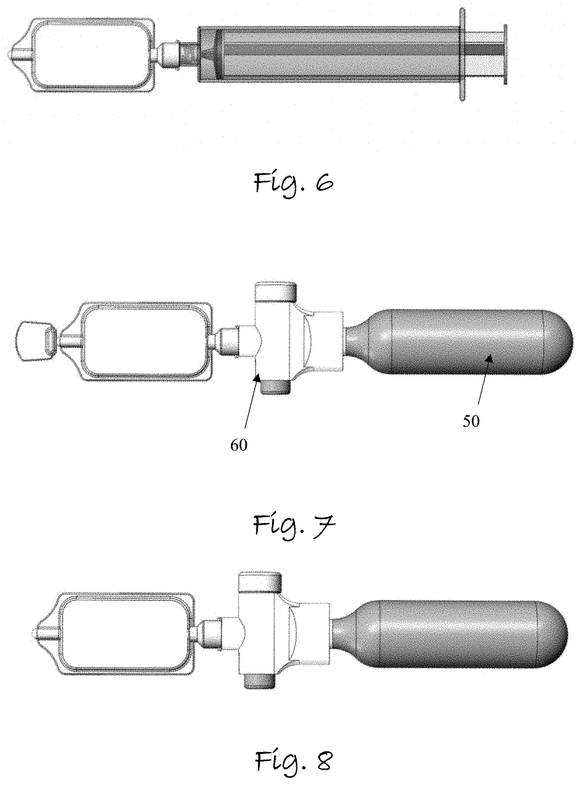

As illustrated in FIGS. 1-3, in FIG. 5 the cover is first remove, revealing at least one orifice of the BFS or FFS. Next, in FIG. 6, the piston compress fluid 11 contained with the syringe.

As described in FIGS. 1-6, the BFS or FFS comprises a breakable membrane, that once the pressure reaches a predetermined value, breaks; thus, enabling the entrance of the pressurized fluid to said BFS or FFS to entrain said substance and deliver the same.

Reference is now made to FIGS. 7-9 illustrating another embodiment of the present invention. In this embodiment, the BFS or FFS is coupled by means of a valve 60 to a container 50, containing pressurized fluid in a predetermined pressured.

As described above, first the BFS' or FFS' cover is removed (see FIG. 8).

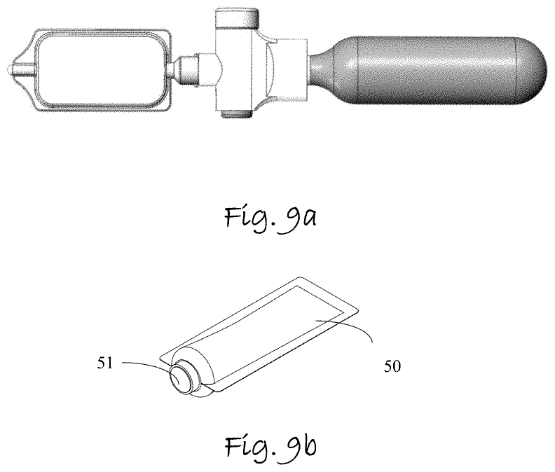

In FIG. 9a, valve 60 is opened. Once said valve is opened, all of the pressurized fluid within the container exits said container and entrain the substance in the BFS or FFS. According to one embodiment, the BFS or FFS, as in the above, comprises a breakable membrane. According to another embodiments, the BFS or FFS is directly coupled to the container only be means of the valve 60, such that when said valve is open ALL the pressurized fluid exit said container and enters said BFS or FFS.

Reference is now made to FIG. 9b, illustrating another embodiment of the present invention in which container 50 (enclosing the pressurized and compressed fluid) is made of material being high barrier film. According to one embodiment the high barrier film is high barrier Aluminum film.

According to this embodiment, the high barrier film (e.g., Aluminum film) is ultrasonic soldered to create the pressurized-fluid container. Such ultrasonic soldering of the Aluminum high barrier film results in an airtight container that prevents any air leakage of the compressed and pressurized gas therewithin.

Such container 50 could be sealed by a breakable membrane 51 (that breaks once the pressure of the pressurized gas, enclosed therewithin, reaches a predetermined level), or septum 51, or any other sealable means.

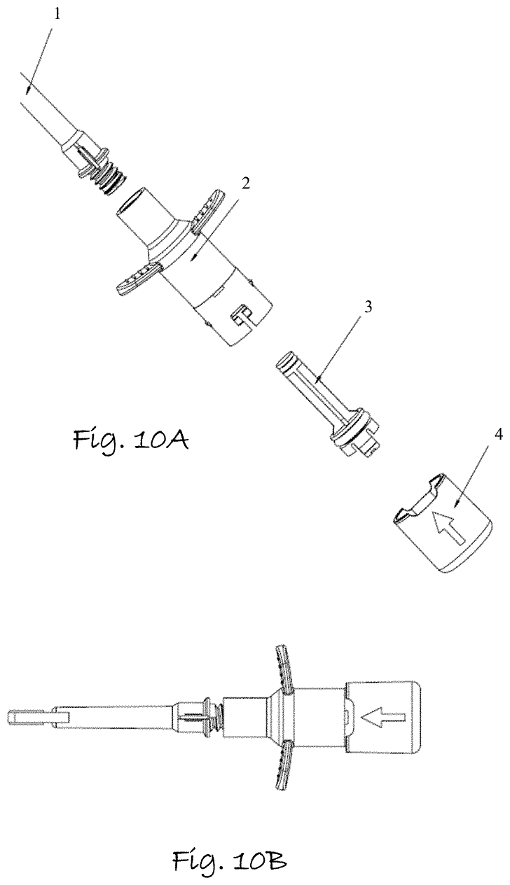

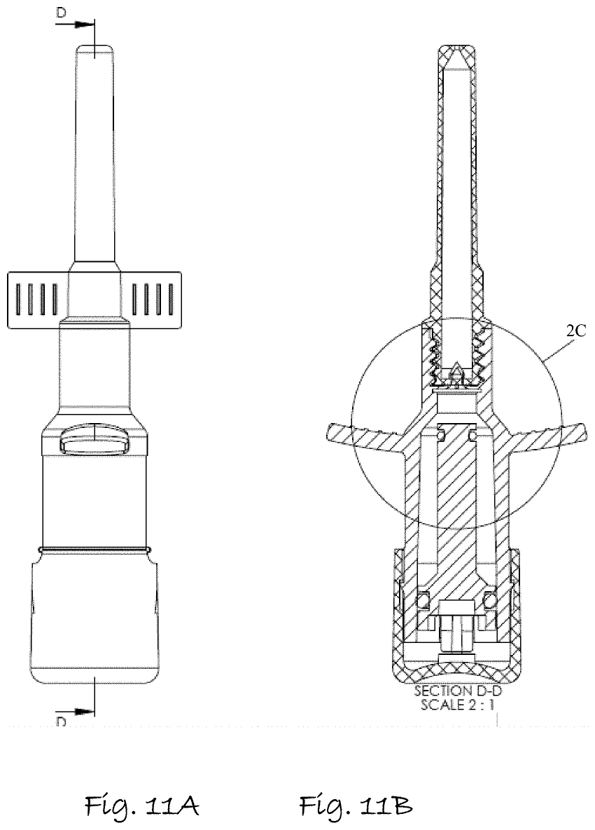

Reference is now made to FIG. 10A-B, disclosing an exemplary device according to one embodiment of the present invention. FIG. 10A shows an exploded view of the device, while FIG. 10B shows the device fully assembled. As shown in FIG. 10A, the device comprises, inter alia, a BFS or FFS nose piece (1), a pressurized-fluid container (2), an air chamber gate (3) and an activation mechanism base (4).

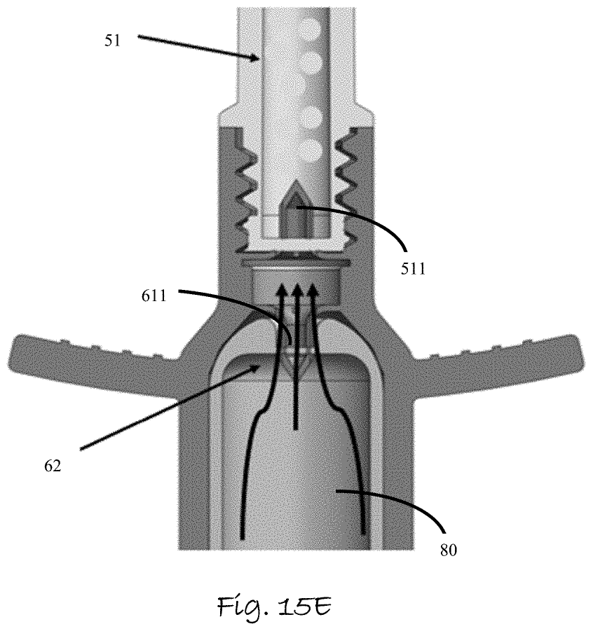

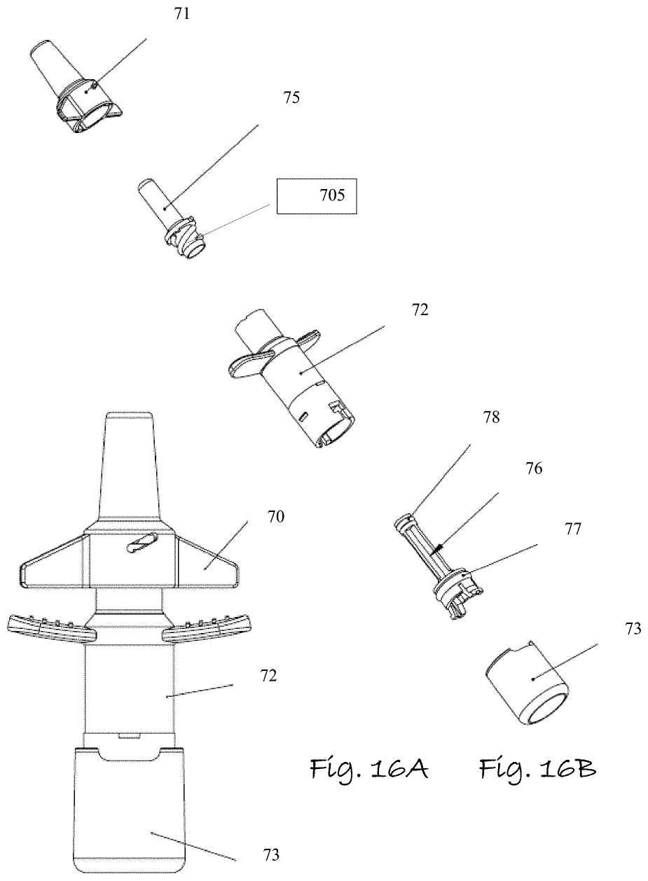

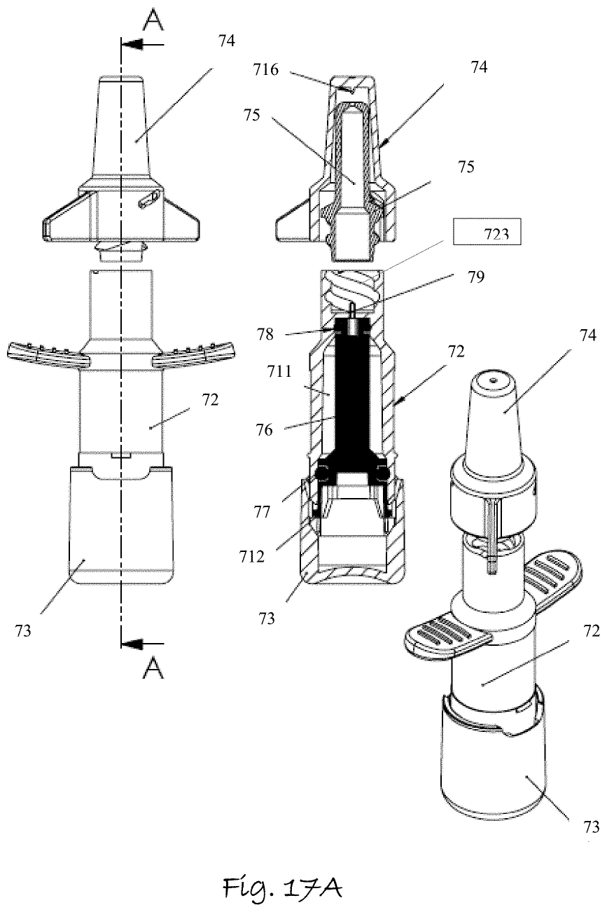

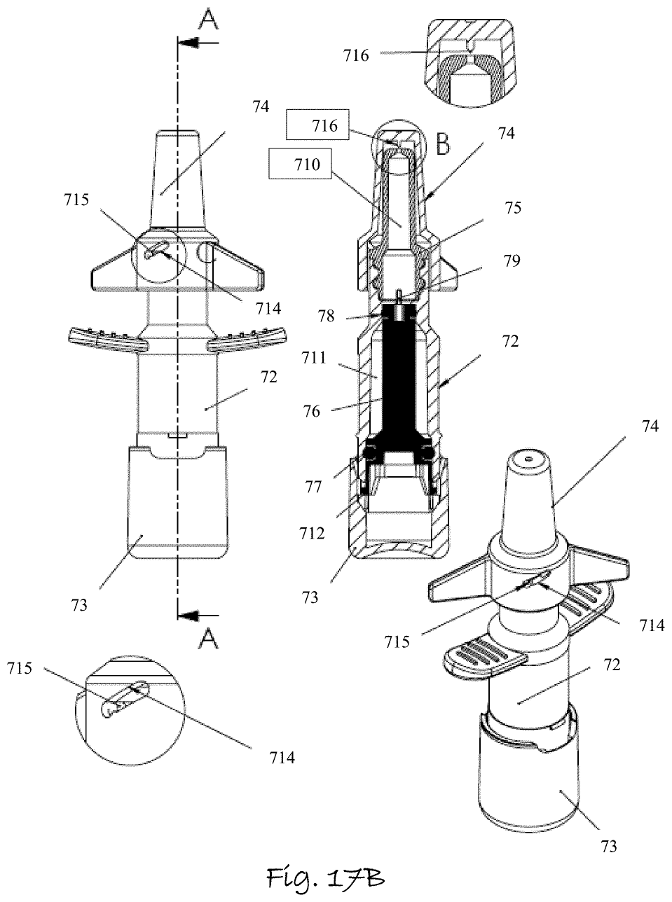

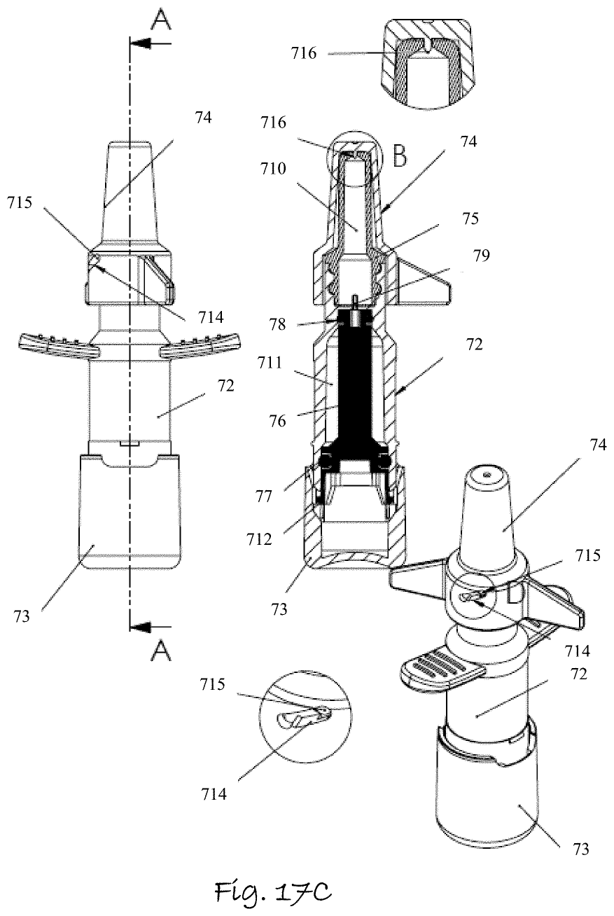

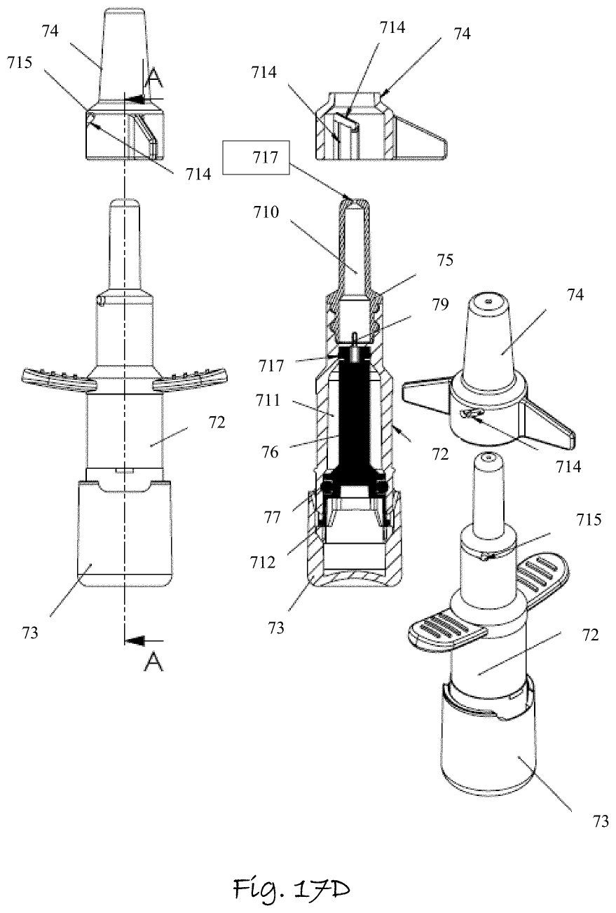

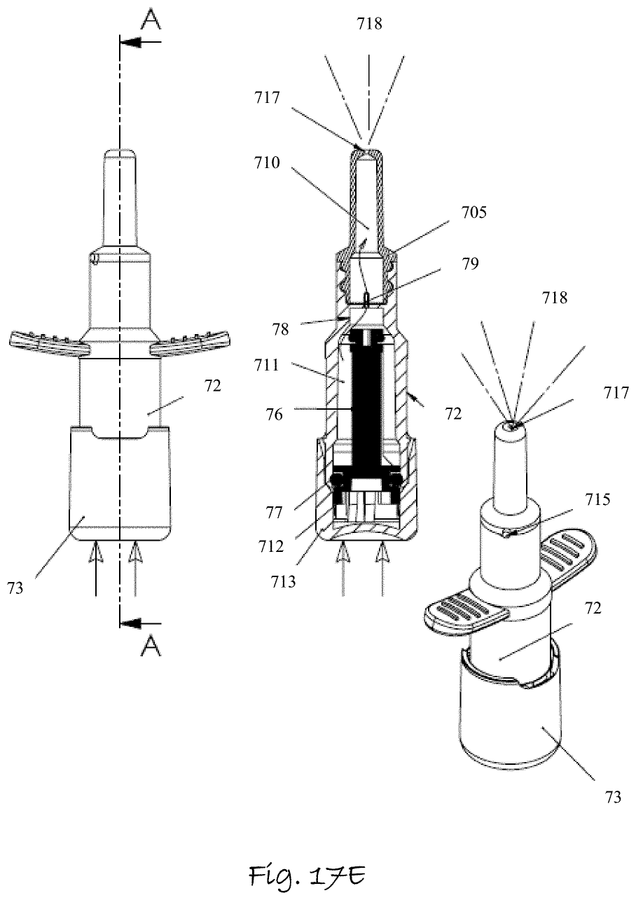

In the FIG. 10A, an air chamber gate (3) has with a first gate O-ring at its proximal end and a second gate O-ring at its distal end (both shown in FIGS. 16A-16B and 17A-17E, as numerical reference 77 and 78).

The pressurized-fluid container (2) will fit over the air chamber gate (3), with the first gate O-ring and the second gate O-ring providing airtight seals before activation so that compressed gas is storable between the air chamber gate (3) and the pressurized-fluid container (2).

As will be disclosed hereinafter, the pierceable drug container (1) (BFS or FFS) in the nosepiece, where there is a puncturing element that punctures the drug container and once the compressed gas is released from the pressurized-fluid container (2), the same entrains the drug and deliver the same to the nasal cavity (see FIGS. 16A-16B and 17A-17E).

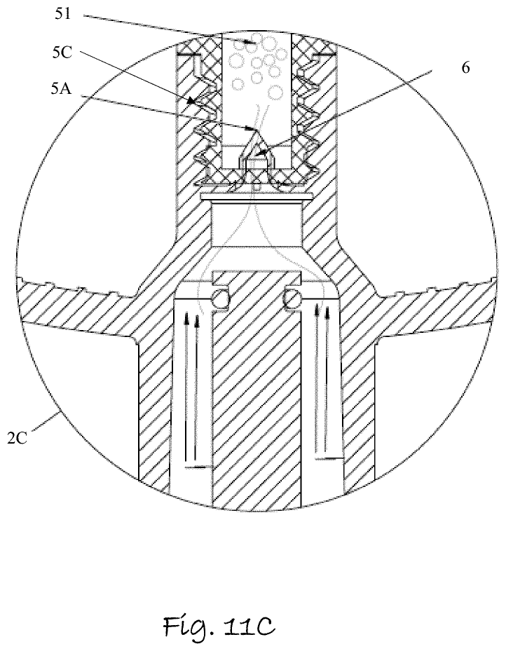

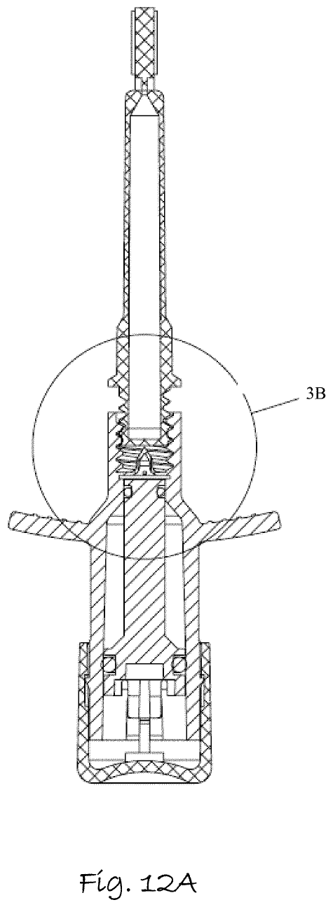

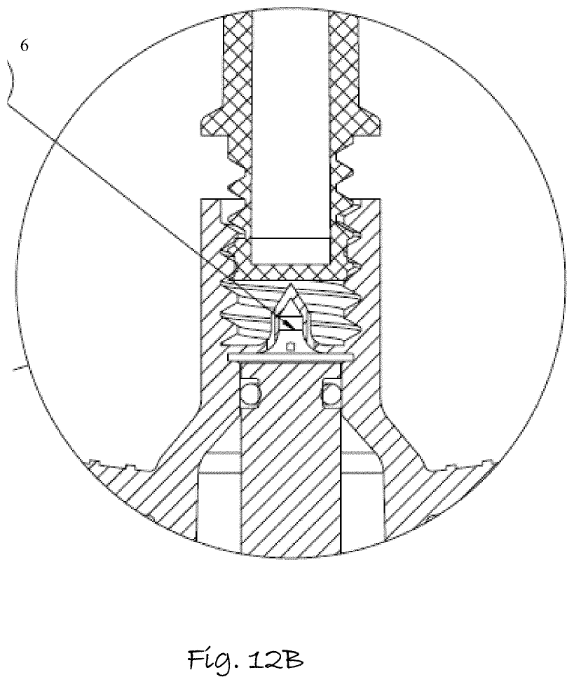

As shown in the Figs. the base of the device forms the activation button (4); to activate, the activation button (4) is pressed upward, then the air chamber gate (3) is drawn downwardly, which removes the sealing of the upper O-ring (78 in FIGS. 16A-16B). The movement of the air chamber gate (3) opens a gap between the pressurized-fluid container (2) and the BFF nose piece (1), allowing the pressurized-fluid to escape from container 2, enter BFF nose piece (1) (after the same has been pierced by the piercing needle 79, shown in FIGS. 16A-16B), and entrain the substance to the nasal cavity. Reference is now made to FIGS. 11A-C, disclosing a device according to another embodiment of the present invention. FIG. 11B depicts a cross section along the line D:D of the device as shown in FIG. 11A. The area within the circle 11C in FIG. 11B is shown enlarged in FIG. 11C, where the device's spike is disclosed (6). Also seen in FIG. 11C is a BSF lower BFS point at which the needle punctures the BFS(5A), BSF nosepiece which contain the drug (51) and an activation screw mechanism (5C).

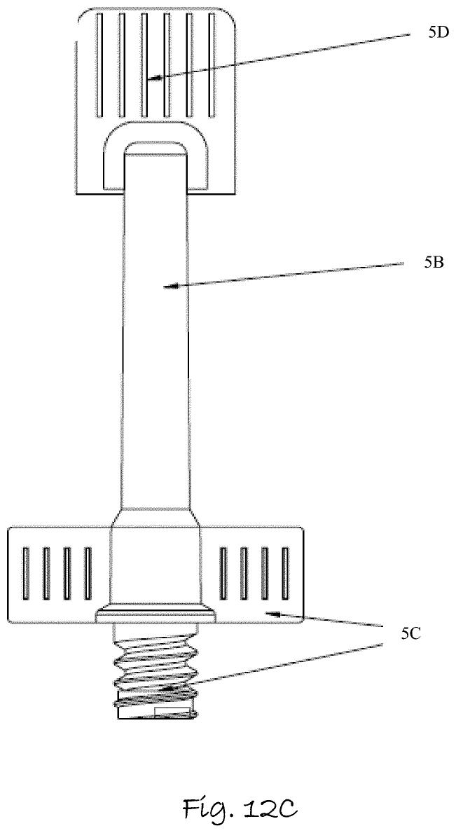

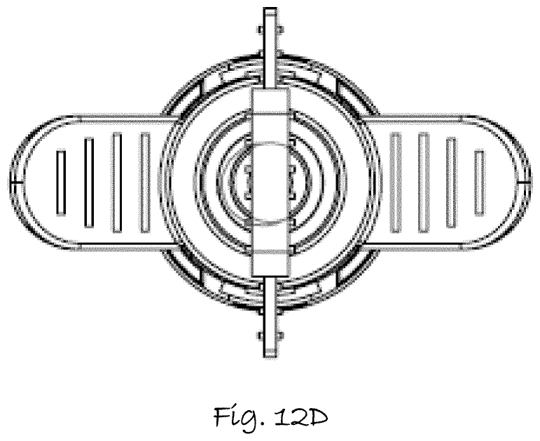

Reference is now made to FIGS. 12A-D, disclosing a device according to another embodiment of the present invention. FIG. 12A shows a cross-section of the device. FIG. 12B shows an enlarged view of the area inside the circle 12B of FIG. 12A. The piercing member (6) can be clearly seen. FIG. 12C shows the exterior of the nosepiece, showing the activation screw mechanism (5C) that is tightened in order to drive the bottom of the drug container against the spike and thereby pierce the drug container; the nosepiece cover (5D) and the main body of the nosepiece (5B). FIG. 12D shows the device from the top.

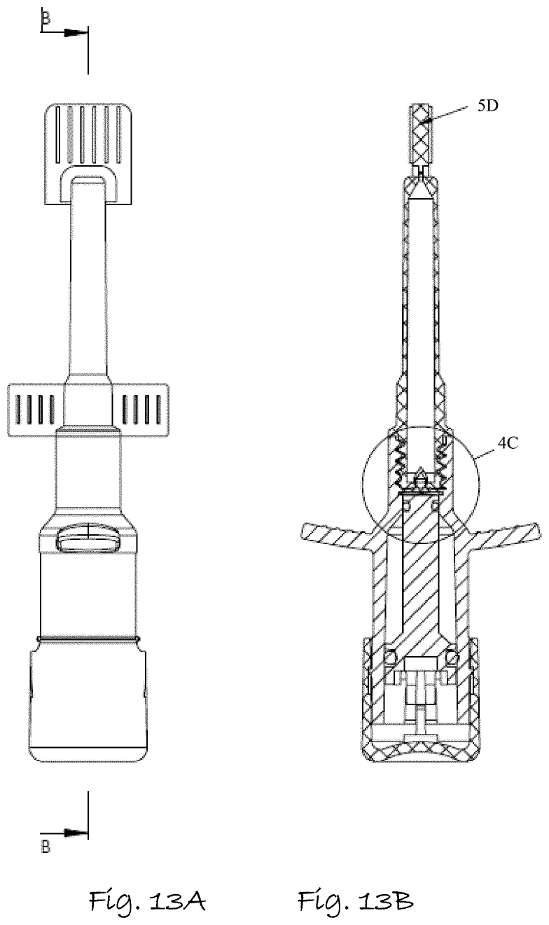

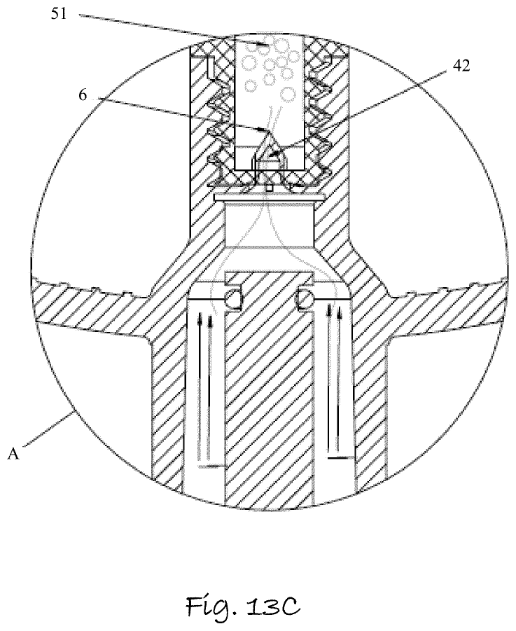

Reference is now made to FIGS. 13A-C, disclosing a device according to another embodiment of the present invention. Here a device is after the activation (FIG. 13A). FIG. 13B shows a cross section of the same along the line B:B. The area within circle 4C is shown enlarged in FIG. 13C, namely a cross section of the piercing member. Drug powder and/or liquid schematically illustrated (51). Air flow through the spike holes (42) entrains the drug.

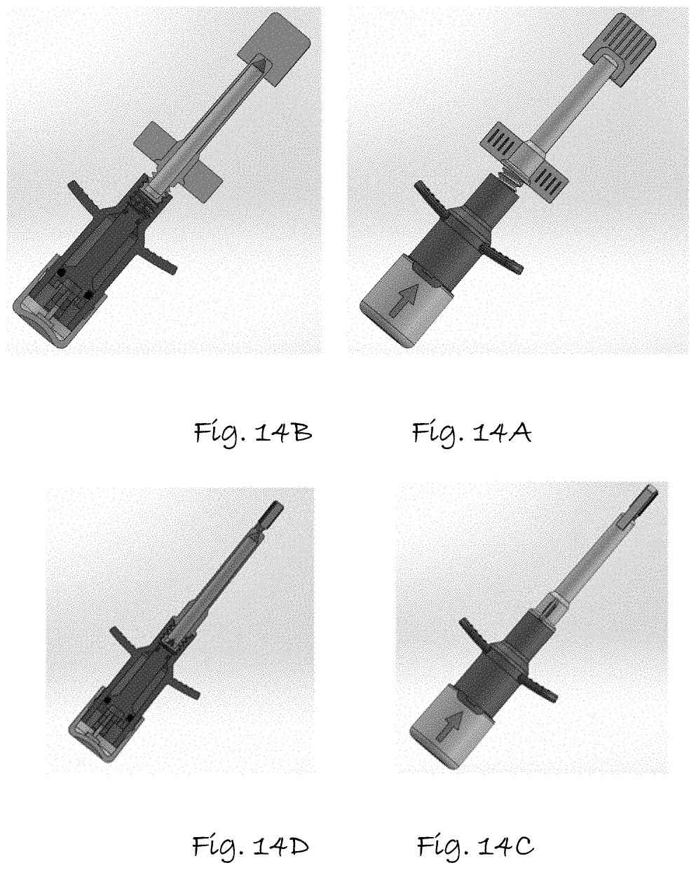

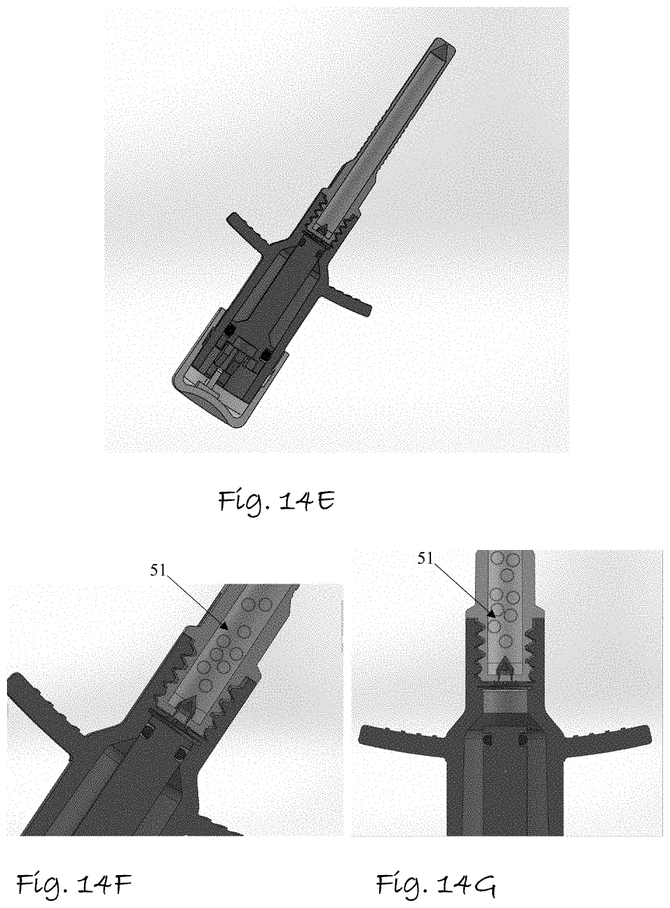

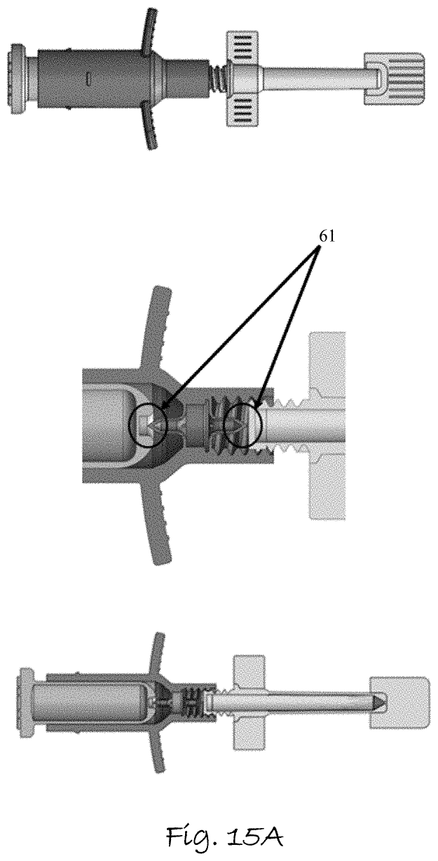

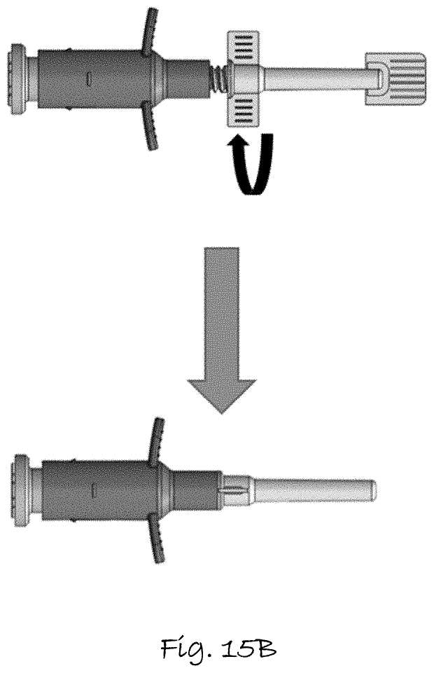

Reference is now made to FIGS. 14A-G, disclosing a device according to another embodiment of the present invention. FIG. 14A is a side view of a pre-used device carrying a BFS or FFS, and FIG. 14B is a cross section of the same. FIGS. 14C and 14D similarly depict the device when connected to a BFS. FIG. 14E shows the same when the device is ready to use, FIG. 14F illustrates the connection between the BFS or FFS to the device; drug (51) is shown. The device after activation presents the flowing drug (51) in FIG. 14G.

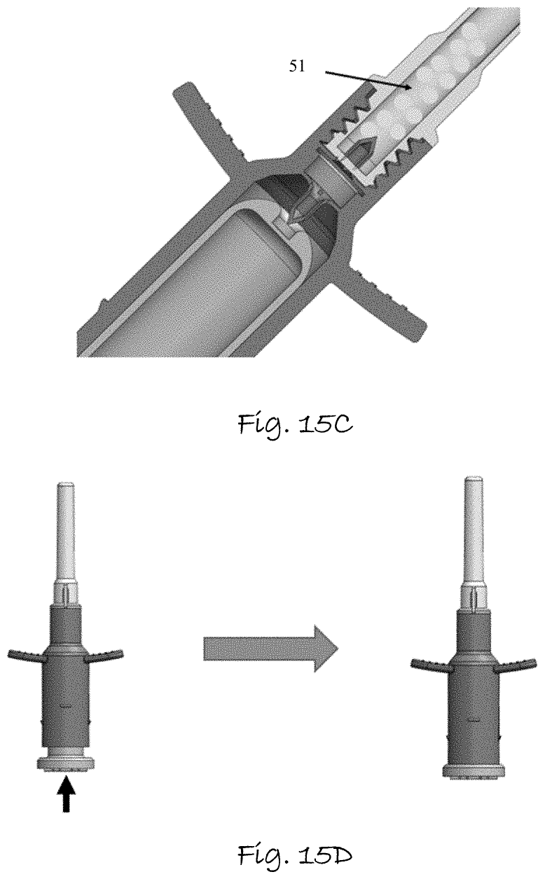

Reference is now made to FIGS. 15A-E disclosing a device according to another embodiment of the present invention. FIG. 15A illustrates a side view (image on the top) of a pre-used device carrying a BFS or FFS. Images on the middle and in the bottom are cross sections of the same, showing BFS or FFS nosepiece and BFS or FFS air container before contact. FIG. 15B shows the second step after introducing the BFS or FFS, namely securing the BFS or FFS to the device, here by turning the nosepiece of the BFS clockwise. Upon rotation of the nosepiece, the piercing member 511 (shown in FIG. 15C), pierces the drug compartment. A further step is removing (e.g., breaking) the cap, the image at the bottom presents the device after breaking the said cap. The drug (51) is presented in cross section view of FIG. 15C. In FIG. 15D, a button at the base of the device is pushed. Such push actuates the base and a second piercing member 611 pierces the container 80. Subsequently, as depicted in FIG. 15E, pressurized fluid (air, nitrogen etc.) flows from its container (62) to the drug-containing BFS or FFS and carries the drug (liquid phase, solid powder particles etc.) (51) outwardly.

One should also note that this example is shown for the same invention but with another kind of pressurized gas container and a different way of compressed gas discharge (by puncturing the container rather than the gate that is shown in the previous figures.

Reference is now made to FIG. 16A, FIG. 16B and FIGS. 17A-17E disclosing a device according to another embodiment of the present invention in a side view and exploded view, respectfully; wherein 70 is a cover holding area; 72 is a pressurized fluid container; 73 is an activation mechanism base; 74 is a cover's body; 75 is a nosepiece; 76 is an air chamber gate; 78 and 77 are O-rings; 79 is a needle; 75 is a nosepiece one way screwing mechanism; 710 is a drug's space; 712 is an air chamber gate's legs; 713 is an air chamber gate's snaps; 714 is a drug storage container locking notch; 715 is a drug storage container locking pin; 716 is an orifice-creating piercing needle; 717 is an orifice; 718 is an aerosol; 720 is a safety latch; 721 and 722 locks; 723 is a pressurized fluid container's internal screwing mechanism. The device comprises modules 70-79 and 710-720, where 71, 74, and 713, and nozzle (orifice) 717 are related with the nosepiece; 72, 76-79, 710-712, 716, to the body and module 73 and 713 is in device's operating button.

In the following sets of figures, namely FIG. 17A-17E, operation modes are illustrated, illustrating a method of utilizing a BFS or FFS technology for delivery substance to a desired target. Furthermore, the present invention utilizes pressurized air to extract said substance from said BFS or FFS (instead of the current method of squeezing thereof). Each pf the figures comprises a front view (left side), cross-section (A:A, middle) and isometric view (right side). FIGS. 17B and 17C further depict a rotation mechanism 714-715, which allows a rotation (here, ¼ rotation) thereby enabled the piercing of the nosepiece substance container.

According to another embodiment, the rotation results in a double piercing of the nosepiece substance container and the pressurized air container.

According to another embodiment the pressurized air container is sealed by means of at least one O-ring, such that movement of the o-ring removes the sealing and enables the release of the pressurized air. In some embodiment at least 2 o-rings are used. One o-ring at the bottom of the pressurized air container and the second at the upper portion of the pressurized air container to seal and separate between the pressurized air container and the nosepiece substance container.

At final step (FIG. 17E), upon pressing the activation mechanism base 73, results in movement of the air chamber gate 76 and the upper o-ring to thereby enable the release of the pressurized air from the pressurized air container and into the nosepiece substance container to entrain the same. Once the pressurized air entrains the substance, aerosol 718 is provided throughout the orifice 717, having a narrow plume angle. It is in the scope of the invention wherein the cover comprises means to protect the drug from UV, e.g., photoprotective agents, such as oxybenzone, titanium oxide and octyl methoxycinnamate.

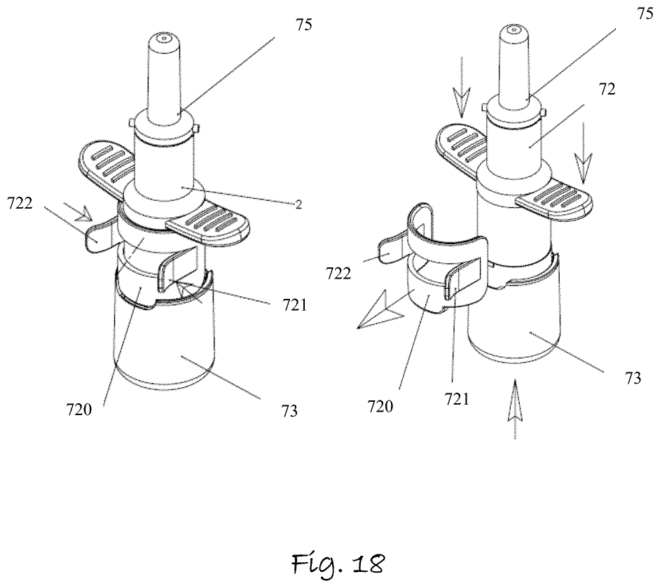

Reference is now made to FIG. 18 disclosing a device according to another embodiment of the present invention, where the device comprises a safety latch 720 with its two locks 721-722, configured to avoid undesired or accidental operation of the device, i.e., by pressing activation mechanism base 73 and pressurized fluid container (body) 72.

It is well in the scope of the invention wherein the pressurized fluid is accommodated within container for a respectively long time, e.g., by having a pre-pressurized container (step 1A) in a fluid connection (step 2A) with the BFS or FFS and releasing the same (step 3A), or alternatively a container suitable to pressuring the fluid in situ within the container, e.g., by introducing a pump or piston mechanism that pressuring ambient air to the container in a first step (step 1B) and accommodating the pressurized fluid along a relatively short time of step 2B, then free the fluid to flow in step 3B.

It is well in the scope of the invention wherein at least one of the steps 1A to 3A, 1B to 3B is provided in an intermitted manner, e.g., by train of n pulses, n is an integer equals to being greater than 2, e.g., 2, 5, 10, 30 or more. Pulses are provided by various mechanisms selected in a non-limiting manner from a series of pressuring efforts (pulsated piston for example and/or series of volume changes within the container); series of releasing pressurized fluid flow, by having rapid open/closed shutting actions of the valve and/or applying blowable lips or rid(s) at the end of the orifice, e.g., as those provided in a mouthpiece of a wind instruments.

The pulses can be identical, e.g., same pressure, same period of time, same volume etc. Additionally, or alternatively, some of pulses can be different by means of e.g., pressure, time, volume etc. It is well in the scope of the invention wherein the fingerprint of the pulses is of increasing pressure, increasing time; and/or increasing pressure decreasing time; and/or decreasing pressure same time and so on and so forth.

According to another embodiment, the present device enables the utilization of BFS or FFS to deliver substances.

According to another embodiment, any device that utilizes pressurized fluid to entrain the substance contained within a BFS or FFS is within the scope of the present invention.

1. A device for delivering one or more substances within at least one body cavity, characterized by at least one pierceable vial comprising V_{sub} [ml or mg] of said substances; said vial having at least one fluid discharging outlet port, configured for placement in proximity to said body cavity; said pierceable vial configured to interface in a sealable manner with at least one pressurized fluid at volume V_{PF} [ml] and pressure P_{PF} [barg];
wherein said device, upon actuation, enables said pressurized fluid to enter into said pierceable vial via at least one said fluid inlet port, entrains said substances, and delivers said substances through said fluid discharging outlet port to within said body cavity in the form of at least one selected from a group consisting of drops, droplets, stream, aerosol and any combination thereof.

2. The device of claim 1, wherein said pressurized fluid is enclosed within at least one pressurized fluid chamber.

3. The device of claim 1, wherein said vial is selected from a group consisting of a pierceable container, a blow-fill-seal and a form-fill-seal, any breakable containers, any puncturable container and any combination thereof.

4. The device of claim 1, additionally comprising an activation mechanism being at least one valve adapted, upon actuation of the same, to enable the entry of said pressurized fluid into said pierceable vial.

5. The device of claim 2, additionally comprising an activation mechanism being at least one valve adapted, upon actuation of the same, to enable the release of said pressurized fluid from said pressurized fluid chamber.

6. The device of claim 2, additionally comprising an activation mechanism being at least one valve adapted, upon actuation of the same, to enable the release of said pressurized fluid from said pressurized fluid chamber and entry of said pressurized fluid into said pierceable vial.

7. The device of claim 1, wherein said vial additionally comprising at least one breakable membrane, such that when the pressure of said pressurized fluid, P_{PF}, reaches a predetermined value, said breakable membrane breaks and enable the entry of said pressurized fluid into said pierceable vial.

8. The device of claim 2, wherein said container additionally comprising at least one breakable membrane, such that when the pressure of said pressurized fluid, P_{PF}, reaches a predetermined value, said breakable membrane breaks and enable the release of said pressurized fluid from said pressurized fluid chamber.

9. The device of claim 1, wherein said container and said vial additionally comprising at least one breakable membrane, such that when the pressure of said pressurized fluid, P_{PF}, reaches a predetermined value, said breakable membrane breaks and enable the release of said pressurized fluid from said pressurized fluid chamber and the entry of said pressurized fluid into said pierceable vial.

10. The device of claim 1, additionally comprising an activation mechanism being at least one piercing element adapted to pierce said pierceable vial.

11. The device of claim 2, wherein said at least one chamber is a container adapted to hold said pressured fluid at said P_{PF} for prolong periods of time.

12. The device of claim 1, wherein said vial is made of material selected from a group consisting of high-density polyethylene and/or polypropylene and any other plastic resin.

13. The device of claim 1, wherein said pressurized fluid is breath exhaled from the user of said device.

14. The device of claim 1, wherein said pressurized fluid is provided by external fluid pump.

15. The device of claim 1, wherein at least one of the following is being held true:
a. said V_{sub} is in a range of about 0.001 to about 7 ml or 0.001 mg to 7 g;
b. V_{PF} is in a range of 0.1 to 50 ml;
c. said P_{PF} is in a range of about 0 to about 10 barg;
d. said at least one fluid inlet port is in a range of 0.01 to 30 mm;
e. said at least one fluid discharging outlet port is in a range of 0.01 to 30 mm;
f. said release of said substances along with said pressurized fluid is performed within a short period of time smaller than 3 seconds;
g. any combination thereof.

16. The device of claim 2, wherein said chamber is a syringe-like device.

17. The device of claim 16, wherein said pressurized fluid is compressed within said chamber by at least one piston.

18. The device of claim 17, additionally comprising at least one piercing element coupled to said piston; said piercing element is adapted to pierce said pierceable vial.

19. The device of claim 2, wherein said chamber is made of material being high barrier film.

20. The device of claim 19, wherein said high barrier film is high barrier Aluminum film.

21. The device of claim 1, wherein at least one of the following is true:
a. said body cavity is selected from a group consisting of nasal cavity, the mouth, the throat, an ear, the eye, the vagina, the rectum, the urethra, and any combination thereof.
b. said pressurized fluid is selected from a group consisting of air, nitrogen, oxygen, carbon dioxide, helium, neon, xenon, nitric oxide and any combination thereof;
c. during dispensing of said at least one substance, a mixture of said predetermined volume V_{gas} [ml] of said pressurized fluid with said predetermined volume V_{sub} [ml or mg] of said substance entrained within it forms a plume of aerosol; said aerosol having a predetermined distribution, said distribution being either homogeneous or heterogeneous, said heterogeneous distribution is selected from a group consisting of: an arbitrary distribution, a distribution in which the density of said at least one substance within said mixture follows a predetermined pattern, and any combination thereof; characteristics of said aerosol selected from a group consisting of: particle size, particle shape, particle distribution, and any combination thereof, are determinable from characteristics of said device selected from a group consisting of: said predetermined volume of said pressurized fluid, said predetermined volume of said substance, said predetermined pressure of said pressurized fluid, said predetermined orifice size, and any combination thereof;
d. at least one said substance is selected from a group consisting of a fluid, a liquid, a powder, an aerosol, a slurry, a gel, a suspension and any combination thereof; and
e. at least one said substance is stored under either an inert atmosphere or under vacuum to prevent reactions during storage.

22. A method for delivering either one or more substances within at least one body cavity, wherein said method comprising steps of:
a. providing a device being characterized by at least one pierceable vial comprising V_{sub} [ml or mg] of said substances; said vial having at least one fluid discharging outlet port; said pierceable vial configured to interface in a sealable manner with at least one pressurized fluid at volume V_{PF} [ml] and pressure P_{PF} [barg];
b. placing said device in proximity to said body cavity; and
c. activating said device; thereby enabling said pressurized fluid to enter into said pierceable vial via at least one said fluid inlet port, and to entrain said at least one substance, and delivered through via said fluid discharging outlet port into said body cavity in the form of at least one selected from a group consisting of drops, droplets, stream, aerosol and any combination thereof.

23. The method of claim 22, wherein said pressurized fluid is enclosed within at least one pressurized fluid chamber.

24. The method of claim 22, wherein said vial is selected from a group consisting of pierceable container, a blow-fill-seal and a form-fill-seal, any breakable containers, any puncturable container and any combination thereof.

25. The method of claim 22, additionally comprising an activation mechanism being at least one valve adapted, upon actuation of the same, to enable at least one selected from a group consisting of (a) enable the entry of said pressurized fluid into said pierceable vial; (b) enable the release of said pressurized fluid from said pressurized fluid chamber; (c) enable the release of said pressurized fluid from said pressurized fluid chamber and entry of said pressurized fluid into said pierceable vial.

26. The method of claim 22, wherein said vial additionally comprising at least one breakable membrane, such that when the pressure of said pressurized fluid, P_{PF}, reaches a predetermined value, said breakable membrane breaks and enable the entry of said pressurized fluid into said pierceable vial.

27. The method of claim 23, wherein said container additionally comprising at least one breakable membrane, such that when the pressure of said pressurized fluid, P_{PF}, reaches a predetermined value, said breakable membrane breaks and enable the release of said pressurized fluid from said pressurized fluid chamber.

28. The method of claim 23, wherein said container and said vial additionally comprising at least one breakable membrane, such that when the pressure of said pressurized fluid, P_{PF}, reaches a predetermined value, said breakable membrane breaks and enable the release of said pressurized fluid from said pressurized fluid chamber and the entry of said pressurized fluid into said pierceable vial.

29. The method of claim 22, additionally comprising an activation mechanism being at least one piercing element adapted to pierce said pierceable vial.

30. The method of claim 22, wherein said at least one chamber is a container adapted to hold said pressured fluid at said P_{PF} for prolong periods of time.

31. The method of claim 22, wherein said vial is made of material selected from a group consisting of high density polyethylene and\or polypropylene and any other plastic resin.

32. The method of claim 22, wherein said pressurized fluid is breath exhaled from the user of said device.

33. The method of claim 22, wherein said pressurized fluid is provided by external fluid pump.

34. The method of claim 22, wherein at least one of the following is being held true:
a. said V_{sub} is in a range of about 0.001 to about 7 ml or 0.001 mg to 7 g;
b. V_{PF} is in a range of 0.1 to 50 ml;
c. said P_{PF} is in a range of about 0 to about 10 barg;
d. said at least one fluid inlet port is in a range of 0.01 to 30 mm;
e. said at least one fluid discharging outlet port is in a range of 0.01 to 30 mm;
f. said release of said substances along with said pressurized fluid is performed within a short period of time smaller than 3 seconds;
g. any combination thereof.

35. The method of claim 23, wherein said chamber is a syringe-like device.

36. The method of claim 35, wherein said pressurized fluid is compressed within said chamber by means of at least one piston.

37. The method of claim 36, additionally comprising at least one piercing element coupled to said piston; said piercing element is adapted to pierce said pierceable vial.

38. The method of claim 22, wherein said chamber is made of material being high barrier film.

39. The method of claim 38, wherein said high barrier film is high barrier Aluminum film.

40. The method of claim 22, wherein at least one of the following is true:
a. said body cavity is selected from a group consisting of nasal cavity, the mouth, the throat, an ear, the eye, the vagina, the rectum, the urethra, and any combination thereof.
b. said pressurized fluid is selected from a group consisting of air, nitrogen, oxygen, carbon dioxide, helium, neon, xenon, nitric oxide and any combination thereof;
c. during dispensing of said at least one substance, a mixture of said predetermined volume V_{gas} [ml] of said pressurized fluid with said predetermined volume V_{sub} [ml or mg] of said substance entrained within it forms a plume of aerosol; said aerosol having a predetermined distribution, said distribution being either homogeneous or heterogeneous, said heterogeneous distribution is selected from a group consisting of: an arbitrary distribution, a distribution in which the density of said at least one substance within said mixture follows a predetermined pattern, and any combination thereof; characteristics of said aerosol selected from a group consisting of: particle size, particle shape, particle distribution, and any combination thereof, are determinable from characteristics of said device selected from a group consisting of: said predetermined volume of said pressurized fluid, said predetermined volume of said substance, said predetermined pressure of said pressurized fluid, said predetermined orifice size, and any combination thereof;
d. at least one said substance is selected from a group consisting of a fluid, a liquid, a powder, an aerosol, a slurry, a gel, a suspension and any combination thereof; and
e. at least one said substance is stored under either an inert atmosphere or under vacuum to prevent reactions during storage.
